(19) United States
(12) Patent Application Publication
(10) Pub. No.: US 2020/0138997 A1
(43) Pub. Date: May 7, 2020

(54) AIR FRESHENER PUMP

(71) Applicant: Matthew De Sanctis, Toronto (CA)

(72) Inventor: Matthew De Sanctis, Toronto (CA)

(21) Appl. No.: 16/182,664

(22) Filed: Nov. 7, 2018

(51) Int. Cl. A61L 9/12

(52) U.S. Cl. CPC A61L 9/122; A61L 2209/111; A61L 2209/15

(57) ABSTRACT

An air freshener apparatus is provided. The air freshener apparatus includes an outer casing, wherein the outer casing includes at least one hole. The air freshener apparatus further includes a fragrance compound housed within the outer casing, configured to scent air coming into contact with the fragrance compound, creating scented air, a blowing apparatus configured to blow the scented air out of the outer casing through the at least one hole, a power supply configured to power the blowing apparatus, and a securing device configured to secure the air freshener apparatus to a surface. The air freshener may be inserted into a shoe, magnetically secured to a metal surface (e.g., a gym locker), and/or clipped lengthwise between a zipper opening (e.g., the zipper on a gym bag).

### CLAIM OF PRIORITY

This application is a United States non-provisional application and claims no priority to any previous United States patent application.

### FIELD OF THE EMBODIMENTS

This invention relates to air fresheners and, in particular, to mobile air-pumping air fresheners configured to be coupled to a surface.

### BACKGROUND OF THE EMBODIMENTS

Air fresheners are commonly placed in easy-to-place open areas, such as rooms and in vehicles. However, air fresheners can also be used for specific applications in which the freshener is in direct vicinity of an object. There are many locations in which unpleasant smells occur that are in areas in which traditional air fresheners are difficult to place. These locations include, e.g., lockers, duffel bags and gym bags, and in shoes. A forced-air air freshener strategically placed in these locations would greatly improve the odors in these locations. For at least these reasons, such an air freshener to solve this problem is needed.

Examples of related art are described below:

U.S. Pat. No. 8,673,223 generally describes a portable powered air freshener that includes an air displacement mechanism carried by a housing including a fan. A scent capsule is carried by the housing and has a chamber containing a fragrant material and a permeable membrane through which a fragrance of the fragrant material can permeate over time.

U.S. Patent Publication No. 2005/0160617 generally describes a bag with active ventilation includes an interior and an exterior defined by at least one panel. The bag includes a fan, which may be solar powered, with an air intake and exhaust, the air intake being disposed toward the exterior and the exhaust being disposed toward the interior. The bag includes at least one conduit fluidly connected to the exhaust for distributing air from the intake to the interior during use. Additionally, the bag may include a bladder having a first panel, a second panel, and a plurality of vents for distributing air to the interior of the bag. The bladder also includes supports that prevent compression of the bladder when a load becomes placed on it, such as when articles become placed in the bag. Thus, the supports allow air to distribute through the bladder to all parts of the bag regardless of the bag contents.

Chinese Patent Publication No. CN201227568Y generally describes a portable minitype air freshener which is used for providing fresh air for people who frequently contact polluted air or venomous and harmful gas. The portable minitype air freshener mainly comprises a filter and a decollator, wherein the filter mainly comprises a filtering unit, a minitype air pump, a power supply, a power supply switch, a shell and an air transmission pipe. The portable minitype air freshener also can be provided with a part having humidifying function, comprising a suction pipe, a small retaining valve and a water receiver. The portable minitype air fresher is a portable air purifying device which is widely applied to indoor and outdoor publics, industrial fields and conditions of fire or leakage of venenous and harmful gases. The utility model perfectly combines various characteristics of dust removal, degerming and peculiar smell removal of a common indoor purifier as well as venenous and harmful gas prevention of an industrial gas mask, and the like, and has the advantages of small volume, light weight, little energy consumption and portability. A bran-new design enables the equipment structure to be more reasonable and realizes the micromation of multifunctional equipment, and the cost for batch manufacture is low.

Chinese Patent Publication No. CN2395444Y generally describes a portable personal negative ion air fresher, comprising a high voltage generator, a negative ion discharge electrode, a battery box, a power supply socket and a mains switch, and the high voltage generator, the negative ion discharge electrode, the battery box, the power supply socket and the mains switch are all arranged in a small plastic main shell body. The negative ion discharge electrode is composed of carbon fibre, the main shell body is composed of a lower plastic shell, an upper plastic shell and a cover of the battery box, and the partial inner cavities of the lower plastic shell and the upper plastic shell and metallic conductive parts are formed into the battery box. Four batteries can be arranged in the battery box with the mains switch, and one end of the battery box is provided with a negative ion air outlet.

Chinese Patent Publication No. CN2233531Y generally describes an integral and portable air freshener. A negative high voltage generator (4) is arranged inside an air freshener body (1). A plug (1) is connected with a power source input terminal (3) of the negative high voltage generator. A discharge point (7) and a pole plate (10) which are arranged on both ends of the air freshener body are connected with a negative high voltage output terminal (6) and an electric discharge loop end (5) respectively of the high voltage generator. Protective hoods (8) and (11) are respectively arranged on the discharge point (7) and the pole plate (10). The air freshener which has the advantages of simple structure, reasonable design, beautiful appearance, small size, and convenient carrying and operation is a novel and practical small household electric appliance.

Chinese Patent Publication No. CN105115061A generally describes a portable air freshener. The portable air freshener comprises an upper housing and a lower housing, wherein the opening of the upper housing faces down, the opening of the lower housing faces up, and the upper housing and the lower housing are oppositely mounted up and down; an air outlet is formed in the top of the upper housing; a blower is arranged at the air outlet in the upper housing; air inlets are formed in the side surfaces of the upper housing; a partition is arranged at the opening of the lower housing; anion generators and plasma generators are mounted at the lower part of the partition; a transformer and a single-chip microcomputer are arranged in the lower housing; the transformer is connected with an external power supply through a connecting piece; and the transformer transmits electricity to the blower, the anion generators and the plasma generators through the single-chip microcomputer after transforming the voltage of the external power supply. The portable air freshener has the benefit that the portable air freshener can be carried about and can be started up to purify air no matter at home or in offices to be good for body health.

Chinese Patent Publication No. CN2483226Y generally describes an air filtering device which comprises a head cover, an air sending pipe and an air filtering device, wherein an air outlet of the air filtering device is connected with the air sending pipe, and the air outlet of the air sending pipe is arranged on the head cover. Owing to the adoption of a portable structure, the utility model has the advantages of reasonable structure, artful design and good performance. The utility model is a novel portable air filtering with portability, convenience and practicability which can get filtered fresh air under the indoor and outdoor conditions of the pollution air environment. The utility model is not only suitable for motorbike riders, but is also suitable for traffic policemen guarding on road and people working in dusty working fields of coal yards, cement factories, environmental sanitation, etc.

Chinese Patent Publication No. CN2341665Y generally describes a portable air freshener which comprises an air freshening machine, an air delivery pipe, a bracket and an air emitting opening. The utility model is characterized in that one end opening of the air delivery pipe is connected with the air freshening machine, and the other end opening of the air delivery pipe is connected with the air emitting opening: the air emitting opening and one part of the air delivery pipe which is connected with the air emitting opening are stably fixed on the bracket. Under the action of the bracket, the clean air delivered by the air emitting opening is dispersed on nose holes or mouths to enable people to breathe and the energy consumption is reduced.

None of the art described above addresses all of the issues that the present invention does.

### SUMMARY OF THE EMBODIMENTS

According to an aspect of the present invention, an air freshener apparatus is provided. The air freshener apparatus includes an outer casing, wherein the outer casing includes at least one hole. The air freshener apparatus further includes a fragrance compound housed within the outer casing, configured to scent air coming into contact with the fragrance compound, creating scented air, a blowing apparatus configured to blow the scented air out of the outer casing through the at least one hole, a power supply configured to power the blowing apparatus, and a securing device configured to secure the air freshener apparatus to a surface.

It is an object of the present invention to provide the air freshener apparatus, wherein the power supply is a battery.

It is an object of the present invention to provide the air freshener apparatus, wherein the battery is a rechargeable battery.

It is an object of the present invention to provide the air freshener apparatus, wherein the air freshener apparatus further includes an indicator light configured to indicate whether the rechargeable battery is charged.

It is an object of the present invention to provide the air freshener apparatus, wherein the air freshener apparatus further includes a charging port for charging the rechargeable battery.

It is an object of the present invention to provide the air freshener apparatus, wherein the securing mechanism is a magnet.

It is an object of the present invention to provide the air freshener apparatus, wherein the magnet is a rare earth magnet.

It is an object of the present invention to provide the air freshener apparatus, wherein the air freshener apparatus further includes a docking station configured to house the outer casing.

It is an object of the present invention to provide the air freshener apparatus, wherein the docking station further includes a charging port for charging the power supply.

It is an object of the present invention to provide the air freshener apparatus, wherein the docking station includes one or more vents.

It is an object of the present invention to provide the air freshener apparatus, wherein the air freshener apparatus further includes a shoe tree into which the outer casing can be inserted.

It is an object of the present invention to provide the air freshener apparatus, wherein the air freshener apparatus further includes one or more clips coupled to an outer surface of the outer casing.

It is an object of the present invention to provide the air freshener apparatus, wherein the one or more clips are configured to secure the air freshener apparatus to a zipper.

It is an object of the present invention to provide the air freshener apparatus, wherein the outer casing includes a bottom casing, a middle casing, and a top casing.

It is an object of the present invention to provide the air freshener apparatus, wherein the air freshener apparatus further includes a light sensor.

It is an object of the present invention to provide the air freshener apparatus, wherein the light sensor is an ambient light sensor.

According to another aspect of the present invention, an air freshener apparatus is provided. The air freshener apparatus includes an outer casing, wherein the outer casing includes at least one hole. The air freshener apparatus further includes a blowing apparatus configured to blow air out of the outer casing through the at least one hole, and a power supply configured to power the blowing apparatus.

It is an object of the present invention to provide the air freshener apparatus, wherein the air freshener apparatus further includes a fragrance compound housed within the outer casing, configured to scent air coming into contact with the fragrance compound, creating scented air.

It is an object of the present invention to provide the air freshener apparatus, wherein the power supply is a battery.

It is an object of the present invention to provide the air freshener apparatus, wherein the air freshener apparatus further includes a securing device configured to secure the air freshener apparatus to a surface.

It is an object of the present invention to provide the air freshener apparatus, wherein the securing mechanism is a magnet.

### BRIEF DESCRIPTION OF THE DRAWINGS

FIG. 1 shows a perspective view of an air freshener apparatus, according to an embodiment of the present invention.

FIG. 2 shows an exploded view of an air freshener apparatus, according to an embodiment of the present invention.

FIG. 3 shows a perspective view of an air freshener apparatus docked in a docking station, according to an embodiment of the present invention.

FIG. 4 shows a perspective view of an air freshener apparatus docked in a shoe tree docking station, according to an embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

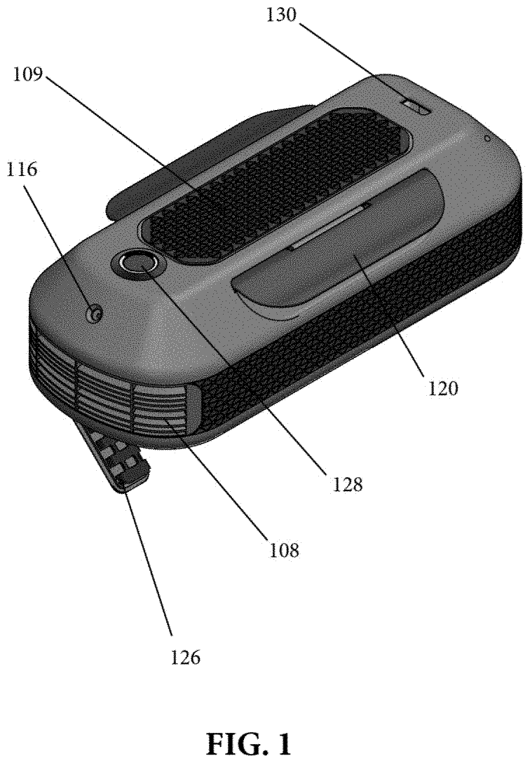

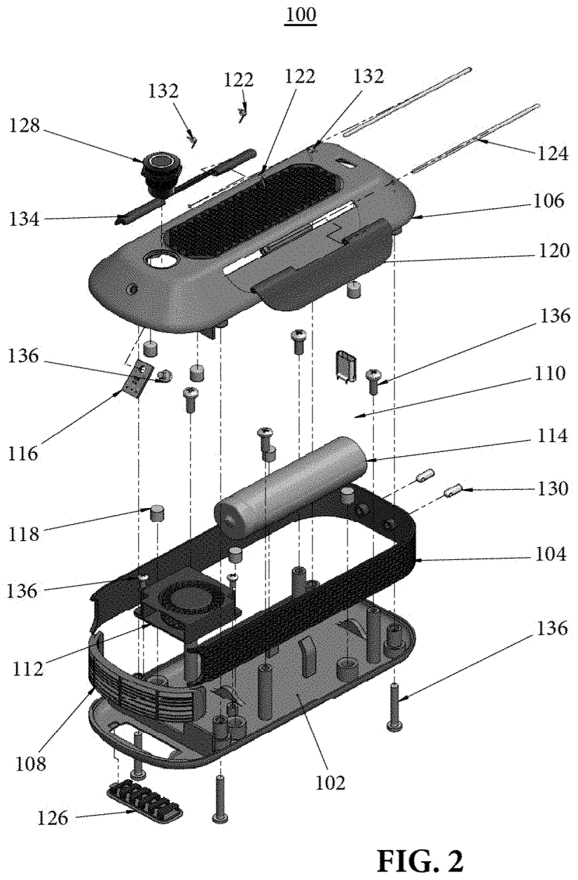

Referring now to FIGS. 1-2, a perspective view (FIG. 1) and an exploded view (FIG. 2) of an air freshener apparatus 100 are illustratively depicted, in accordance with an embodiment of the present invention.

According to an embodiment, the air freshener apparatus 100 includes a bottom casing 102, a middle casing 104, a top casing 106, a grill 108, a printed circuit board (PCB) assembly 110, a blower 112, a battery 114, a light sensor 116, one or more magnets 118, a clip 120, a clip spring 122, a clip pin 124, a scent disc holder 126, a push button switch 128, a charging port 130, a mirror clip spring 132, a mirror clip 134, and/or any other suitable components. According to an embodiment, one or more of the components of apparatus 100 are held together via one or more screws 136. It is noted, however, that other means of holding the parts together may also be used, while maintaining the spirit of the present invention. According to some embodiments, the apparatus 100 may further includes an inlet grill 109.

According to an embodiment, a scent disc is inserted into the scent disc holder 126. The scent disc provides a pleasant fragrance, scenting the air. It is noted, however, that other means of scenting the air, other than a scent disc, may also be used, while maintaining the spirit of the present invention. According to an embodiment, the blower 112 blows the scented air through the grill 108, which acts as a vent. This freshens the air in proximity to the air freshening apparatus 100.

According to an embodiment, the magnet 118 is a rare earth magnet (e.g., a neodymium magnet). It is noted, however, that other suitable types of magnet may also be used, while maintaining the spirit of the present invention. According to an embodiment, the one or more magnets 118 are positioned on the bottom casing 102. The one or more magnets 118 enable the apparatus 100 to become magnetically coupled to a metallic surface. For example, the apparatus 100 may be positioned within a locker (e.g., a gym locker), enabling the apparatus to be positioned to effectively freshen the scent of the locker. It is noted, however, that the apparatus may be positioned at any suitable location, whether being magnetically coupled or not, while maintaining the spirit of the present invention.

According to an embodiment, the apparatus 100 is battery 114 powered, enabling the apparatus 100 to be placed in locations in which an external power supply is not available or difficult to connect to. According to an embodiment, the battery 114 is rechargeable.

According to an embodiment, the clip 120 can be pulled up and, when released, retracted to its original state. This enables a user to use the clip 120 to fasten the apparatus 100 to an object along a length of a zipper.

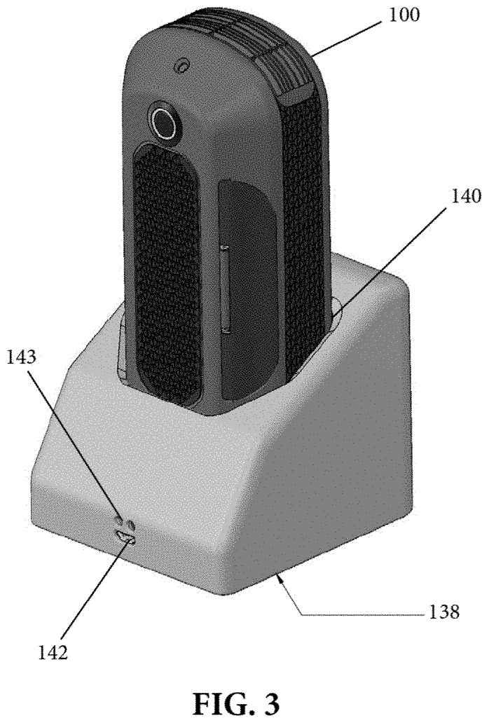

Referring now to FIG. 3, a perspective view of the air freshener apparatus 100 docked in a docking station 138 is illustratively depicted, in accordance with an embodiment of the present invention.

According to an embodiment, the docking station 138 is configured to house the air freshener apparatus 100. According to an embodiment, the docking station 138 is configured to charge the battery 114. According to an embodiment, the docking station 138 includes an opening 140 into which the apparatus 100 is inserted.

According to an embodiment, there is a power adapter (not shown) which plugs into the charging port 130 of the apparatus 100. According to an embodiment, the docking station 138 includes one or more power ports 142 into which a power supply is connected. According to an embodiment, the power from the power supply runs through the power port 142 and to the charging port 130 of the apparatus 100. According to an embodiment, the docking station 138 may further include one or more indicator lights 143 configured to indicate the charge of the apparatus 100.

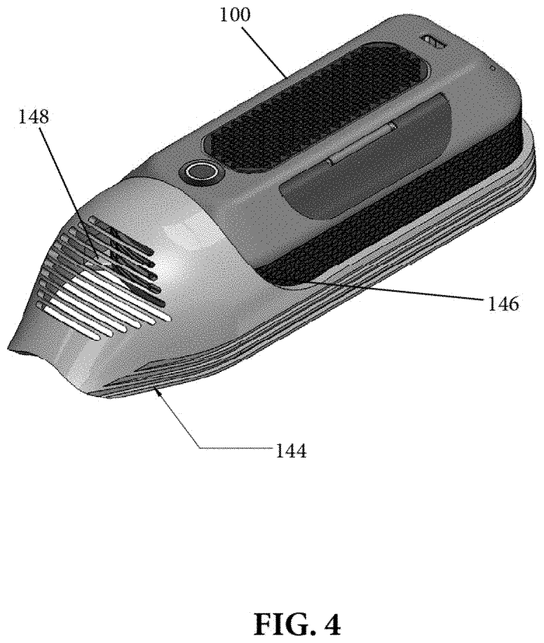

Referring now to FIG. 4, a perspective view of the air freshener apparatus 100 docked in a shoe tree docking station 144 is illustratively depicted, in accordance with an embodiment of the present invention.

According to an embodiment, the shoe tree docking station 144 is configured to be positioned within a shoe. According to an embodiment, the shoe tree docking station 144 includes an opening 146 into which the apparatus 100 is inserted. According to an embodiment, the shoe tree docking station 144 further includes one or more vents 148 enabling air forced by the blower 112 to pass through.

According to an embodiment, the apparatus 100 is inserted into the shoe tree docking station 144. The combined apparatus 100 and shoe tree docking station 144 is then inserted into a shoe, allowing the apparatus 100 to pump scented air into the shoe, aiding in the elimination of undesirable odors in the shoe. According to an embodiment, the shoe tree docketing station 144 does not have charging capabilities. According to an embodiment, the shoe tree docking station 144 has charging capabilities.

When introducing elements of the present disclosure or the embodiment(s) thereof the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. An air freshener apparatus, comprising:
an outer casing,
wherein the outer casing includes at least one hole;
a fragrance compound housed within the outer casing, configured to scent air coming into contact with the fragrance compound, creating scented air;
a blowing apparatus configured to blow the scented air out of the outer casing through the at least one hole;
a power supply configured to power the blowing apparatus; and
a securing device configured to secure the air freshener apparatus to a surface.

2. The air freshener apparatus as recited in claim 1, wherein the power supply is a battery.

3. The air freshener apparatus as recited in claim 2, wherein the battery is a rechargeable battery.

4. The air freshener apparatus as recited in claim 3, further comprising an indicator light configured to indicate whether the rechargeable battery is charged.

5. The air freshener apparatus as recited in claim 3, further comprising a charging port for charging the rechargeable battery.

6. The air freshener apparatus as recited in claim 1, wherein the securing mechanism is a magnet.

7. The air freshener apparatus as recited in claim 6, wherein the magnet is a rare earth magnet.

8. The air freshener apparatus as recited in claim 1, further comprising a docking station configured to house the outer casing.

9. The air freshener apparatus as recited in claim 8, wherein the docking station further includes a charging port for charging the power supply.

10. The air freshener apparatus as recited in claim 8, wherein the docking station includes one or more vents.

11. The air freshener apparatus as recited in claim 1, further comprising a shoe tree into which the outer casing can be inserted.

12. The air freshener apparatus as recited in claim 1, further comprising one or more clips coupled to an outer surface of the outer casing.

13. The air freshener apparatus as recited in claim 1, wherein the one or more clips are configured to secure the air freshener apparatus to a zipper.

14. The air freshener apparatus as recited in claim 1, wherein the outer casing includes a bottom casing, a middle casing, and a top casing.

15. The air freshener apparatus as recited in claim 14, wherein the light sensor is an ambient light sensor.

16. An air freshener apparatus, comprising:
an outer casing,
wherein the outer casing includes at least one hole;
a blowing apparatus configured to blow air out of the outer casing through the at least one hole; and
a power supply configured to power the blowing apparatus.

17. The air freshener apparatus as recited in claim 16, further comprising:
a fragrance compound housed within the outer casing, configured to scent air coming into contact with the fragrance compound, creating scented air.

18. The air freshener apparatus as recited in claim 16, wherein the power supply is a battery.

19. The air freshener apparatus as recited in claim 16, further comprising:
a securing device configured to secure the air freshener apparatus to a surface.

20. The air freshener apparatus as recited in claim 19, wherein the securing mechanism is a magnet.
